(19) United States
(12) Patent Application Publication
(10) Pub. No.: US 2015/0140054 A1
(43) Pub. Date: May 21, 2015

(54) Scent/fragrance sampling card and method of manufacture

(71) Applicant: John DiGREGORIO, Brick, NJ (US)

(72) Inventor: John DiGREGORIO, Brick, NJ (US)

(21) Appl. No.: 13/998,646

(22) Filed: Nov. 21, 2013

(51) Int. Cl. A61L 9/12; A01N 25/08

(52) U.S. Cl. CPC A61L 9/12; A01N 25/08
USPC 424/411; 239/57; 493/328

(57) ABSTRACT

A scent delivery system in the form of a scent sampling card in which a fragrance/oil of a scented nature is encapsulated in the form of microscopic beads or scent of scented oil encased in a gelatinous or plastic cell wall, the encapsulation protects the microscopic beads and the scent or fragrance from escaping or migrating out of the card until its intended time of use, and holds both ply closed by bonding to both halves. The card comprises a two ply substrate which sandwiches the area of encapsulated fragrance within. The two ply substrate can be formed of paper stock, or paper stock and another substance, such as a laminate. Once the card is opened, the encapsulation is penetrated, and the capsules of fragrance and scent are ruptured or break open exposing and emitting the fragrance.

### RELATED APPLICATIONS

Applicant claims the benefit of provisional application Ser. No. 61/736,773, filed Dec. 13, 2012.

### BACKGROUND OF THE INVENTION

1. Field of the Invention

The present invention relates to a scent/fragrance sampling card in which a scented or fragrance oil is microencapsulated, then applied to another substrate, where it is sandwiched between two outer protective layers, the separation of the outer protective layers causing the release of the fragrance or scent from the microencapsulated fragrance oil or scent infused or coated substrate.

2. Description of the Prior Art

Manufacturers of consumer products have a profound interest in determining the acceptability of their products by the consuming public. For that reason, manufacturers routinely test market new products with consumers in order to get their reactions. The test marketing can involve a myriad of trials. The manufacturer can hire representatives to offer samples of the new products at the locations where the new products might be sold. These samples would be provided gratis to consumers and then the consumer would be asked their reaction. Additionally, the manufacturer might have a mass mailing of small samples of the new product to consumers in a particular geographical location, together with a questionnaire regarding the consumer's likes or dislikes with respect to the product.

The products involved in such consumer testing can range from health care products, such as hair shampoos and conditioners, to makeup, to kitchen cleaners, all purpose cleaners, dishwashing cleaners, clothes and soap blends, and health care, as well as beauty care products. The manufacturers are interested in not only how the consumer reacts to the new product and its effectiveness, but also if the consumer is influenced by the style and even the scent or fragrance of the product. Thus health care and beauty care products are test marketed extensively. An individual's most common interaction with a test marketing situation involving a scent or fragrance occurs at the perfume or mens cologne counter at large department stores. There are numerous sample bottles of cologne, perfume, and eau de toilette on display for sampling. In some instances, these are actual bottles of the product with small absorbent substrates associated therewith where one can spray the scent of a particular product onto the absorbent substrate, and wave the substrate and then smell the substrate to obtain a sense of the fragrance or scent of the particular perfume. In other instances, the manufacturers go to great cost and expense to have extremely small samples of the product prepared which can be given away as promotional materials to people in order to gain their feedback regarding the fragrance and scent.

With respect to health care, the most common form of fragrance or scent is that of a spray inhalant available primarily for use as a nasal decongestant, however, these products are bulky and not easily carried on an individual's person. This scent/fragrance sampling card of the present invention could be utilized for the inclusion of health care products in the form of nasal decongestants, germ fighters, or other formulas intended to combat against flu and cold germs, bacteria, and sickness. The particular health care product could be positioned again in a microencapsulated fragrance oil, or onto a scented infused or coated substrate which is sandwiched between two impervious plies of protective material which maintains the efficacy of the health care product until the outside protective plies have been separated, which in turn would expose and release the encapsulated health care product either infused in oil or scent infused or coated substrate.

### OBJECTS OF THE INVENTION

An object of the present invention is to provide for a novel scent/fragrance delivery system for inexpensively providing sample scents and fragrances to the consumer for sampling purposes.

A still further object of the present invention is to provide for a novel scent/fragrance delivery system in the form of a sampling card which can be infused with microencapsulated oils or a scent infused or coated substrate protected by an outer layer which serves to release a beauty care product and/or scent or fragrance upon opening.

A still further object of the present invention is to provide for a novel scent/fragrance delivery system in the form of a sampling card in which health care products in the form of anti-germ, anti-cold, anti-bacterial, or anti-flu defenses can be positioned in a microencapsulated oil or scent infused or coated substrate which will release the health care product upon the separation of the protective outer layers covering the protective oils or scent infused substrate.

A still further object of the present invention is to provide a novel scent/fragrance delivery system in the form of a sampling card for aroma therapy formulas for, but not limited to, purpose of energy, concentration, stress release, sleep, or dietary scents to help relieve appetite.

A still further object of the present invention is to provide for a novel scent/fragrance sampling card which can be easily constructed and fabricated using a variety of different printing processes.

A still further object of the present invention is to provide for a novel scent/fragrance delivery system in the form of a sampling card which is convenient for travel and convenient for an individual to carry on his or her person.

A still further object of the present invention is to provide for a novel scent/fragrance delivery system in the form of a sampling card which has a long shelf life.

A still further object of the present invention is to provide for a novel scent/fragrance sampling card or health delivery card which is of a convenient size and which does not make the user self-conscious during the course of its use.

### SUMMARY OF THE INVENTION

A scent delivery system in the form of a scent sampling card in which a fragrance/oil of a scented nature is encapsulated in the form of microscopic beads or scent of scented oil encased in a gelatinous or plastic cell wall, the encapsulation protects the microscopic beads and the scent or fragrance from escaping or migrating out of the card until its intended time of use. The card comprises a two ply substrate which sandwiches the area of encapsulated fragrance within. The two ply substrate can be formed of paper stock, or paper stock and another substance, such as a laminate. Once the card is opened, the encapsulation is penetrated, and the capsules of fragrance and scent are ruptured or break open exposing and emitting the fragrance. The delivery system may be used as a single use or reusable system depending upon the method of securing the two ply substrate.

### BRIEF DESCRIPTION OF THE DRAWINGS

These and other objects of the present invention will become apparent, particularly when taken in light of the following illustrations wherein:

FIG. 1 is a top open view of the scent/fragrance delivery system sampling card of the present invention;

FIG. 2 is a perspective view of the scent/fragrance delivery system sampling card;

FIG. 3 is a schematic of a narrow web flexographic printing press layout for the manufacture of the scent/fragrance delivery system sampling card;

FIG. 4 is a planar layout view of a half width printing press impression for the manufacture of the scent/fragrance delivery system sampling card;

FIG. 5 is an alternative layout for the manufacture of the scent/fragrance delivery system sampling card; and

FIG. 6 is a perspective view of a folding and die cutting layout for the manufacture of the scent/fragrance delivery system sampling card.

### DETAILED DESCRIPTION OF THE INVENTION

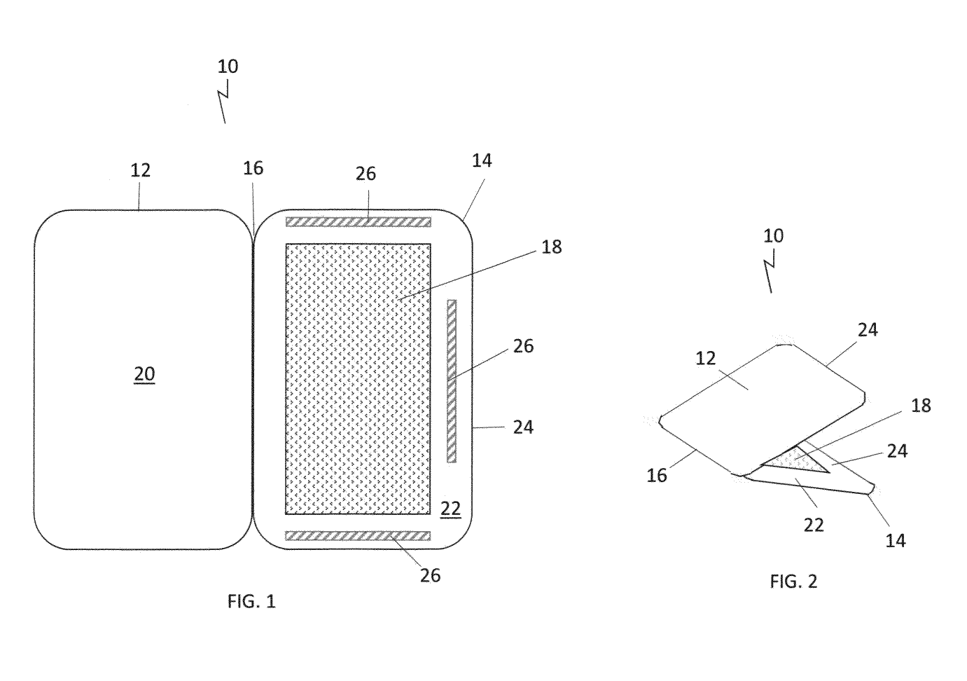

FIG. 1 is a top open view of the scent/fragrance delivery system sampling card 10 of the present invention, hereinafter sampling card. Sampling card 10 is formed of a first upper ply 12 and a lower ply 14 joined and folded along a common edge 16. Plies 12 and 14 can both be of paper stock or upper ply 12 could be fabricated from a laminate. Microscopic beads of a scented oil encased in a gelatinous or plastic medium 18, is applied to lower ply 14 of sampling card 10 initially as a liquid, which then bonds with the inner sides 20 and 22 of both top ply 12 and bottom ply 14 when the two plies are folded upon themselves along folded edge 16. The encapsulated fragrance 18 bonds to both plies 12 and 14 as it dries in the closed orientation. This bonding protects the encapsulated oils 18 and prevents their migration from the sampling card 10 and further prevents any introduction of an outside scent or fragrance to the bonded layer. Once the sampling card 10 is opened by grasping the edge 24 opposed to the folded edge, and separating the two plies 12 and 14, and pulling them open, the encapsulation is ruptured and the encapsulated scent or fragrance is exposed thereby emitting its fragrance. In the configuration thus explained, the sampling card would be used as a single use sampling card, discardable once it is opened. The option of including an area of hot melt glue, or some other non-permanent glue or adhesive 26 at locations indicated on the lower ply 14, would allow for the resealing of sampling card 10 after its initial opening for further reuse until the scent and/or fragrance had been exhausted.

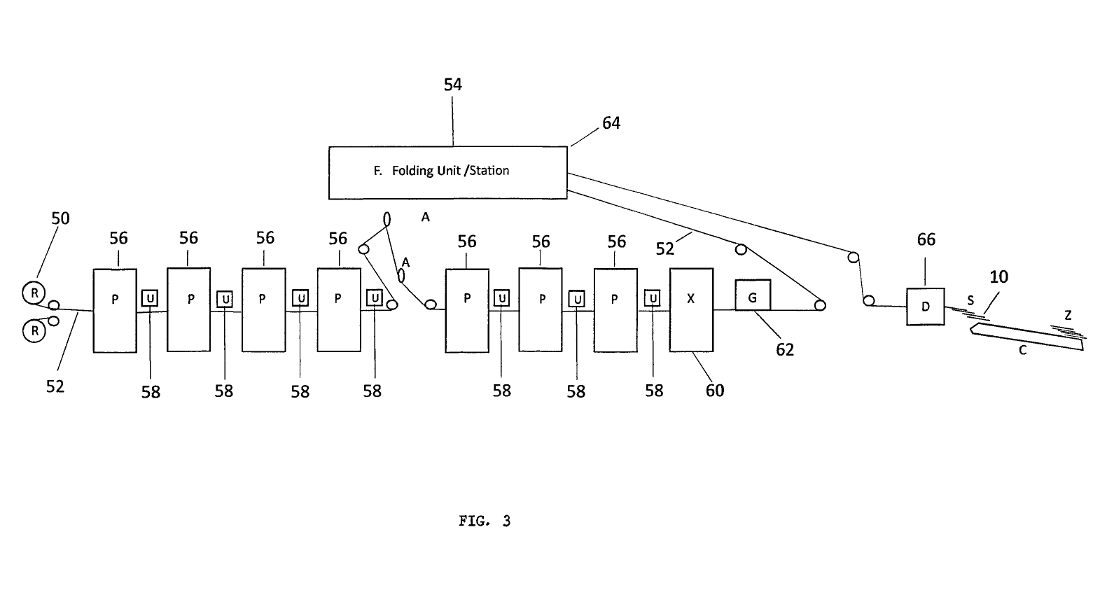

FIG. 2 is a perspective view of the sampling card 10 in a die cut and folding orientation. It should be noted that there is a marginal area 30 surrounding fragrance area 18 to ensure there is no migration nor escape of fragrance and allows the card to be opened by peel page along edge 24.

The sampling card 10 has been described with respect to the illustrations of FIGS. 1 and 2 which clearly illustrate that the sampling card is approximately the size of a standard credit card, and has approximately the same shape. It will be recognized that the size and shape of the sampling card is variable depending upon its intended use. As a scent sampling card for manufacturer's products, such as a perfume or cologne, the credit card shape might be appropriate for mass production and advertising. However, the shape of the sampling card 10 can vary depending upon its purpose. For instance, if a certain food company wished to promote their produce, they might choose to have sampling cards prepared in the shape of a fruit with that fruit's fragrance encapsulated between the plies of the card.

The scent sampling card 10 can be manufactured by several processes. The concerns are the requirement of less waste, and reducing the amount of additional production stages in order to finish the scent sampling card. With respect to short run quantities of product, a small printing press, such as a narrow web flexographic printing press layout, can be utilized to produce the product completely in a single pass. Alternatively, a large press with format design can be utilized to reduce the number of multi-stage operations in order to obtain a scent sampling card. FIG. 3 is a schematic diagram of the use of a small printing press, such as a narrow web flexographic printing press arrangement. Rolls of stock 50, which in this case for purposes of explanation would be paper stock, is introduced as a web 52 into the printing press layout 54. A series of printing units 56, are alternatively utilized to print the desired graphics or indicia and the color of same. If ultraviolet inks are used, high intensity ultraviolet drying lamps 58 would be positioned between each successive printing station so as to dry the ink previously applied before entering the successive printing station 56. Once the printing on the outside surface of plies 12 and 14 has been completed and dried, the web 52 would enter a fragrance or scent application unit 60 wherein the encapsulated oil beads in the gelatinous or plastic medium, would be deposited in a web format, into the inner surface of the lower ply 14. The web would then if so desired, enter into a glue station 62 where the glue 26 as illustrated in FIG. 1, would be applied to the margins of lower ply 14. The web would then proceed to a folding station 64 where the web would be folded upon itself such that the upper ply 12 and the lower ply were folded along common edge 16, thus sandwiching the encapsulated oil and gelatinous or plastic medium 18 between the two plies 12 and 14. The web 52 would then continue on to a die cutting unit or station 66 where the web would be continuously die cut producing the finished sampling cards 10. In this method the width of web 52 would be slightly greater than the width of sampling card 10 illustrated in FIG. 1 so that top ply 12 is folded onto lower ply 14 thereby minimizing waste at die cut station 66.

The fragrance oil or scent which is encapsulated may be selected from a wide variety of stock fragrances, oils, or made as a special order. Fragrance oils or scents are formulated with as little volatile or water soluble reactive ingredients as possible to help enable encapsulation which is to form a wall around the fragrance oil creating beads of the microencapsulated fragrance. The fragrance solution which resembles a slurry, contains a transporter fluid to help transfer the encapsulated fragrance onto the printable substrate. Once the fragrance slurry has cured and the transporter solution has been flashed off or dissipated, only the fragrance of the oil itself will remain and provide an accurate rendition of the fragrance once the capsules have been broken. The microencapsulated fragrance will bond to the substrate it is transferred to and remain intact until, as in this case, the scent sampling card is opened along the opposing edge from common edge 16. The scent is thereby revealed.

For larger production quantities of the scent sampling cards, larger printing equipment would be more efficient, such as, but not limited to, heat-set web offset printing, which will yield more product in a shorter amount of time. Web printing refers to paper or other printable substrates loaded onto/into the printing press to provide a steady stream or web of paper, unlike sheet-fed printing, which uses sheets of paper for production instead of rolls of paper.

Multiples of scent sampling cards may be stepped or ganged as used in the art in printing plates of large presses, which will mass produce cards at a higher yield per impression. The impression is determined by the amount of area across the width of the press and the circumference or distance around the printing plate cylinder. For example, a “half web” press circumference may be 17¾ inches and the width may be 26 inches.

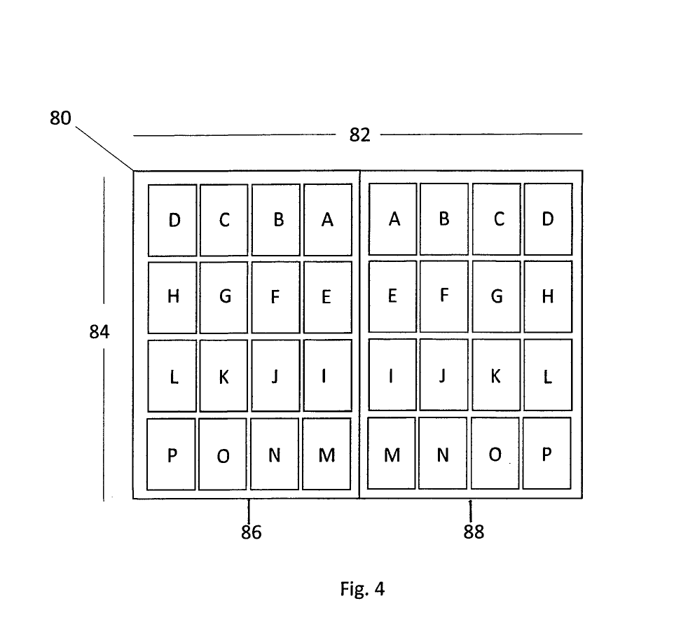

Considering a “half web” press, a 4×4 layout will yield 16 card units per each single press impression. This is based on a “half web” press circumference of 17.750 inches and a width of 26 inches. This will produce a 16 card layout with each revolution of the printing press. This layout or array is illustrated in FIG. 4. The web 80 of paper stock would be 26 inches in width 82 and the impression on the rotating cylinder would be 17.750 inches in circumference yielding a length 84 of 17.750 inches. The rotating cylinder would contain the necessary indicia for impressing the web with the array illustrated in two half impressions. The left half impression 86 contains 16 impressions labeled A through P, and the right half impression 88 contains 16 impressions similarly labeled A through P, the left half impression and the right half impression being mirror images of each other. Impressions A through P on the left half 86 would represent the inside top ply of the scent sampling card 10 and the right half 88 impressions A through P would represent the inside bottom ply of the scent sampling card. It will be recognized that the web 80 may be subjected to a plurality of press rollers of a 17 inch diameter to apply the necessary inks required to obtain the necessary color and printing indicia desired on the sampling cards. Dependent upon the type of ink utilized, ultraviolet drying lamps may be required as with respect to the flex print method of FIG. 3, or a high heat dryer utilizing forced hot air for alternative inks might be used.

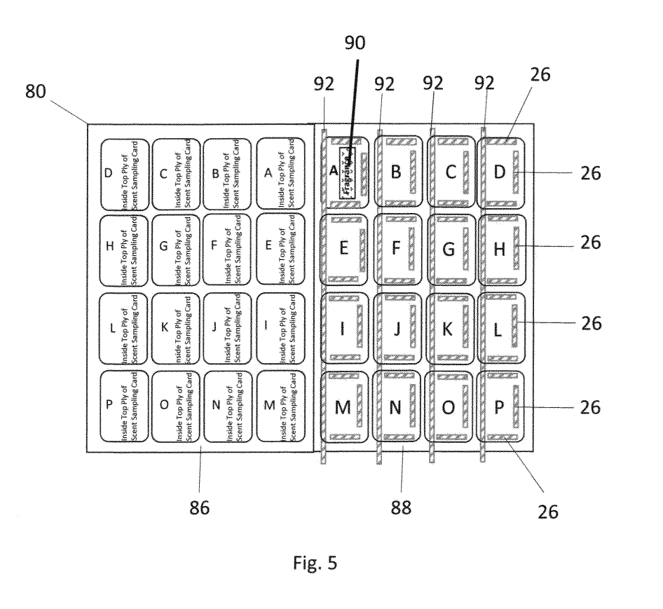

Once the ink/graphics have been printed and dried, the paper is primed to receive a fragrance which can be applied from a separate unit or by a method of extrusion through a nozzle. In one method, the encapsulated fragrance in the form of a slurry would be introduced into a pan or reservoir from which it is drawn out by a roller and transferred to the web 80. This method can be achieved by applying the fragrance using separate pads or patches affixed around the circumference of the cylinder in as may rows across the cylinder as required to accommodate the layout, the pads or patches positioned so as to register with each impression on the right half 88 side of the web as indicated in FIG. 5 wherein the location of a patch of fragrance 90 has been indicated. Similar patches would be positioned on all of the impressions, A through P on the right half 88 of the web. An alternative method would be to utilize a single plate that has multiple pad/patch areas composited on the plate and positioned in accordance with the layout or array as illustrated in FIG. 4.

An alternative method of applying the fragrance patch would be to have a plurality of extrusion nozzles positioned across the web in alignment with the array of right half 88 impressions 1 through 4 and subsequent rows thereafter. As the web passed below the extrusion nozzles, whose width would determine the width of the fragrance patch, the nozzles would extrude the slurry direction onto all of the impressions on the right half of the web.

In the half web press array as illustrated in FIGS. 4 and 5, the web is ultimately going to be folded upon itself to form the two plies of the scent sampling card. In most instances the fragrance patch 90 would be sufficient to seal the two plies, however, there may be a need for a hingable edge to be formed with respect to the subsequently produced cards which will serve the purpose of the common hinge edge 16 as illustrated in FIG. 1. In order to accomplish this, a plurality of gluing nozzles in alignment with the right hand 88 impression of the web would be positioned such that a permanent glue would be extruded in a continuous line 92 along the left hand edge of the impressions A through P on the right half 88 of the web 80 which formed the inside of the bottom ply. As illustrated hereafter, this line of glue when folded with the left hand impression will form the equivalent of hinge 16 in scent sampling card 10. The additional glue areas 26 as illustrated in FIG. 1 are positioned the same on the impressions A through P on the right half impression of the web. These would be applied in a similar manner.

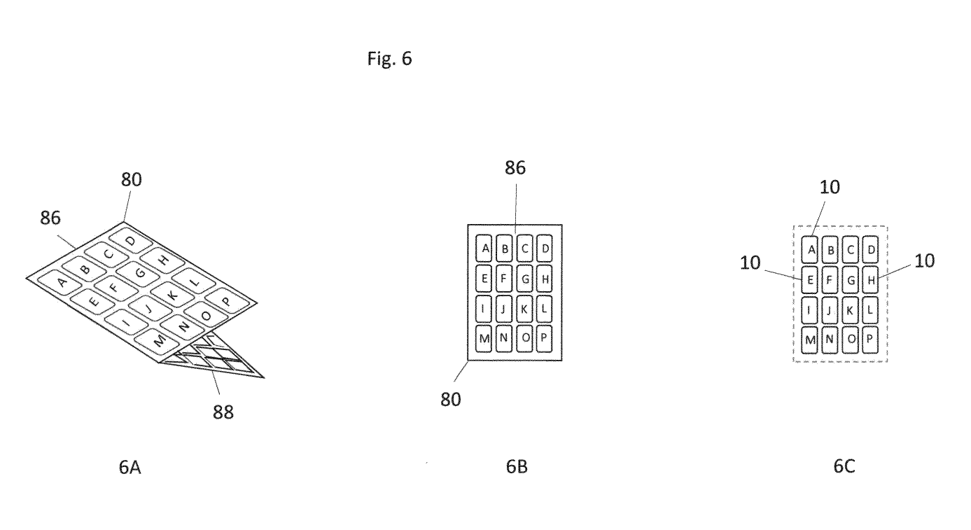

FIG. 6 is a three step process illustrating the method of folding and die cutting the array that is illustrated in FIGS. 4 and 5. FIG. 6A illustrates the folding of the web from left to right bringing into registration the impressions A through P on the left hand side 86 of the web 80, which represent the inside top ply of the sampling card. These are brought into registration with the impressions A through P on the right half 88 of the web which signifies the inside bottom ply of the scent sampling card. The various glues 92 and 26 if required and fragrance patch 90 have already been applied. The resultant fold results in 16 scent sampling card within a matrix as illustrated in FIG. 6B. The web 80, now having been reduced to half its width as a result of the fold is cut by a plurality of cutting knives in a linear direction to form the size matrix as illustrated in FIG. 6B, that is approximately 13 inches in width and 17.750 inches in length. This matrix is then die cut in a method known in the art as illustrated in FIG. 6C which results in 16 scent sampling cards 10 in the configuration as originally illustrated in FIG. 1.

In the methods described, it has been identified that the fragrance patch in most instances would be sufficient to secure the two plies of the scent sampling card together until separated. The preferred method of separation would be the grasping of the two plies along a longitudinal edge, and pulling apart the two plies with one's fingers. It will be recognized that the sampling card might also be manufactured in the method by which an embossed perforation is formed along one edge, preferably longitudinal edge, of the scent sampling card, this embossed perforation presenting a tear away perforation removable by the user which would then allow for more facile grasping and separation of the plies to release the fragrance or scent.

Therefore, while the present invention has been disclosed with respect to the preferred embodiments thereof, it will be recognized by those of ordinary skill in the art that various changes and modifications can be made without departing from the spirit and scope of the invention. It is therefore manifestly intended that the invention be limited only by the claims and the equivalence thereof.

I claim:

1. A scent sampling card for the delivery of a scent, fragrance, or anti-bacterial germicide, the scent sampling card comprising:
a first ply substrate and a second ply substrate of equal size, said first ply substrate comprising the top substrate and said second ply substrate comprising of the bottom substrate, the bottom substrate having coated thereon a fragrance area comprising microscopic beads of fragrance, scent, or scented oil, encased in said fragrance area, said fragrance area upon drying sufficient to bond said first top ply to said second bottom ply and encasing said fragrance area.

2. The scent sampling card in accordance with claim 1 wherein said first top ply substrate and said second bottom ply substrate are formed from a single substrate, said first top ply substrate and said second bottom ply substrate having a common edge allowing said top ply substrate and said bottom ply substrate to be folded upon each other, thereby encapsulating said fragrance area, allowing said fragrance area to bond said plies.

3. The scent sampling card in accordance with claim 2 wherein said common edge of said first top ply and said second bottom ply is slit allowing the first top ply substrate to be positioned on said bottom ply substrate encapsulating said fragrance area and allowing said fragrance area to bond said plies.

4. The scent sampling card in accordance with claim 1 wherein said first top ply substrate and said second bottom ply substrate are separable along an edge of said scent sampling card, said separation releasing said fragrance, scent or scented oil from said fragrance area.

5. The scent sampling card in accordance with claim 1 wherein a non-permanent adhesive patches may be positioned about said periphery of said second bottom ply substrate comprised of a hot melt glue or other non-permanent adhesive allowing for the resealing and reusability of said scent sampling card.

6. The scent sampling card in accordance with claim 1 wherein said first ply substrate and said second ply substrate are formed from paper stock.

7. The scent sampling card in accordance with claim 1 wherein said first ply substrate and said second ply substrate are formed from paper stock and a laminate such as but not limited to a polymer film.

8. A method of fabricating a scent sampling card which employs an encapsulated scent, fragrance, or anti-bacterial germicide for release upon opening of said scent sampling card, the method comprising the following steps:
(a) positioning a roll of paper stock in alignment with a printing press;
(b) unwinding said paper roll into a paper web and passing said paper web through a plurality of print rolls thereby forming an indicia and coloring on said paper web identifying an upper ply and lower ply of a scent sampling card;
(c) drying said inking on said paper stock;
(d) positioning a slurry of fragrance or scent in the form of a reservoir into registration with a portion of said paper web;
(e) transferring a portion of said slurry of fragrance or scent onto said web in the form of a fragrance area in registration with a ply of said sampling card;
(f) folding said paper web in half longitudinally thereby encasing said fragrance area between said folded paper web in registration with said upper ply and said lower ply;
(g) allowing said slurry to dry and cure, bonding said upper and lower ply; and
(h) die cutting said folded web to form successive scent sampling cards defined by the indicia and coloring of step (b) and having said defined fragrance area in accordance with step (e).

9. The method in accordance with claim 8 wherein adhesive patch may be applied in registration to said upper or lower ply and about said fragrance area between steps (e) and (f) for additional bonding.

10. The method in accordance with claim 9 wherein said adhesive patches comprise a hot glue non-permanent adhesive allowing for resealing and reusability of said scent sampling card.

11. The method in accordance with claim 8 wherein said paper roll is formed of a narrow web and subjected to flexographic printing presses allowing the side by side fabricating of a top ply and bottom ply forming said scent sampling card, said application of said fragrance area and said adhesive patches being to said bottom ply, said top ply being secured to said bottom ply during said folding process.

12. The manufacture process in accordance with claim 8 wherein said fabrication is accomplished by a half web press in which said upper ply and said bottom ply are formed in 16 card arrays, the web, when folded upon itself, forming 16 scent sampling cards, prior to die cutting to form 16 separate scent sampling cards.
